(19) United States
(12) Patent Application Publication
(10) Pub. No.: US 2023/0223143 A1
(43) Pub. Date: Jul. 13, 2023

(54) PREDICTING TEMPORAL IMPACT OF INTERVENTIONS BY DECONVOLVING HISTORICAL RESPONSE DATA

(71) Applicant: International Business Machines Corporation, Armonk, NY (US)

(72) Inventors: Aisha Walcott, Nairobi (KE); Jonathan Lenchner, North Salem, NY (US); OSNAT BAR-SHIRA, Givatayim (IL); Michal Rosen-Zvi, Jerusalem (IL)

(21) Appl. No.: 17/573,809

(22) Filed: Jan. 12, 2022

(51) Int. Cl. G16H 50/20; G16H 50/70

(52) U.S. Cl. CPC G16H 50/20; G16H 50/70

(57) ABSTRACT

A method including: receiving multiple time series of data, each time series representing a health-related temporal impact of a historical set of elements on an adverse health-related condition; automatically deconvolving the health-related temporal impact, to determine an individual contribution of each element of each of the historical sets to the health-related temporal impact of the respective historical set, wherein the deconvolving is performed respectively of an additive impact and/or a multiplicative impact of elements; receiving a selection of a new set of elements which is different from any one of the historical sets; and based on the determined individual contributions, automatically predicting a temporal impact of the new set of elements, wherein the new set consists of elements selected from said group but which do not jointly constitute any one of the historical sets.

### BACKGROUND

The invention relates to the field of health, industrial, agricultural, and other applications of signal and data processing techniques.

In such applications, it is often desired to predict the outcome (or “impact”) of a set of multiple actions that are being carried out concurrently. Such prediction, naturally, involves exploration of available data regarding how the underlying system (e.g., a pandemic, an industrial process, or agricultural crop yield) has reacted to similar actions in the past.

However, in many scenarios, the precise set of desired actions has never been tested before, and there is no historical data from which one can readily deduce the impact of that set. Rather, the historical data may consist of demonstrated outcomes of other sets of actions, which only partially overlap those actions in the desired set, or do not overlap them at all. For example, if the set of desired actions consists of actions A, B, C, D, and E, the historical data may only be evident of the outcomes of sets such as A+C+D, B+C+E, A+X, B+C+D+Y, A+B+C+D+E+Z, X+Y, and so on. Deducing the predicted outcome of the set A, B, C, D, and E from such historical data is not straightforward. This problem may become even more severe when the possible outcomes are not a simple numerical value, but rather involve a temporal aspect to them, such as a response to the actions which evolves as a function of time.

The foregoing examples of the related art and limitations related therewith are intended to be illustrative and not exclusive. Other limitations of the related art will become apparent to those of skill in the art upon a reading of the specification and a study of the figures.

### SUMMARY

The following embodiments and aspects thereof are described and illustrated in conjunction with systems, tools and methods which are meant to be exemplary and illustrative, not limiting in scope.

One embodiment relates to a computer-implemented method comprising: receiving multiple time series of data, each time series of data representing a health-related temporal impact of a historical set of elements on an adverse health-related condition, wherein the elements are selected from the group consisting of: health-related interventions, and health-related risk factors; receiving an indication of whether there are two or more elements in any of the historical sets that are estimated to jointly have additive impact within the respective set; automatically defining all non-selected elements as elements that have multiplicative impact within their respective sets; automatically deconvolving the health-related temporal impact of each of the historical sets, to determine an individual contribution of each element of each of the historical sets to the health-related temporal impact, wherein said deconvolving is performed respectively of the additive impact and multiplicative impact; receiving a selection of a new set of elements which is different from any one of the historical sets; and based on the determined individual contributions, automatically predicting a temporal impact of the new set of elements, wherein the new set consists of elements selected from said group but which do not jointly constitute any one of the historical sets.

Another embodiment relates to a system comprising: (i) at least one hardware processor; and (ii) a non-transitory computer-readable storage medium having program code embodied therewith, the program code executable by said at least one hardware processor to: receive multiple time series of data, each time series of data representing a health-related temporal impact of a historical set of elements on an adverse health-related condition, wherein the elements are selected from the group consisting of: health-related interventions, and health-related risk factors; receive an indication of whether there are two or more elements in any of the historical sets that are estimated to jointly have additive impact within the respective set; automatically define all non-selected elements as elements that have multiplicative impact within their respective sets; automatically deconvolve the health-related temporal impact of each of the historical sets, to determine an individual contribution of each element of each of the historical sets to the health-related temporal impact, wherein said deconvolving is performed respectively of the additive impact and multiplicative impact; receive a selection of a new set of elements which is different from any one of the historical sets; and based on the determined individual contributions, automatically predict a temporal impact of the new set of elements, wherein the new set consists of elements selected from said group but which do not jointly constitute any one of the historical sets.

A further embodiment relates to a computer program product comprising a non-transitory computer-readable storage medium having program code embodied therewith, the program code executable by at least one hardware processor to: receive multiple time series of data, each time series of data representing a health-related temporal impact of a historical set of elements on an adverse health-related condition, wherein the elements are selected from the group consisting of: health-related interventions, and health-related risk factors; receive an indication of whether there are two or more elements in any of the historical sets that are estimated to jointly have additive impact within the respective set; automatically define all non-selected elements as elements that have multiplicative impact within their respective sets; automatically deconvolve the health-related temporal impact of each of the historical sets, to determine an individual contribution of each element of each of the historical sets to the health-related temporal impact, wherein said deconvolving is performed respectively of the additive impact and multiplicative impact; receive a selection of a new set of elements which is different from any one of the historical sets; and based on the determined individual contributions, automatically predict a temporal impact of the new set of elements, wherein the new set consists of elements selected from said group but which do not jointly constitute any one of the historical sets.

In some embodiments, said deconvolving comprises applying gradient descent optimization to the health-related temporal impact of each of the historical sets, by: (a) for each element in the respective historical set, generating a sigmoid function which is based on a guessed impact value and a guessed response time value of the respective element, (b) combining the sigmoid functions of all elements in the respective set into a composite response function which represents the respective set, wherein said combining comprises multiplication and addition of the sigmoid functions, in correspondence to the multiplicative impact and the additive impact, respectively, (c) calculating a difference between the composite response function and the received health-related temporal impact of the respective historical set, (d) iterating steps (a) through (c) with a different guessed impact value and a different guessed response time value in each iteration, until the calculated difference is reduced to or below a predetermined threshold, and/or until further gradient descent is no longer possible, (e) defining the different guessed impact value and the different guessed response time of the last iteration as the determined individual contribution of each element of the respective historical set to the health-related temporal impact of the respective historical set.

In some embodiments, said predicting of the temporal impact of the new set of elements comprises multiplying and/or adding the determined individual contributions of those elements included in the new set of elements, wherein the multiplying and/or adding correspond to the multiplicative impact and/or the additive impact, respectively.

In some embodiments, the method further comprises, or the program code is further executable to, prior to said deconvolving, automatically normalize the multiple time series of data to a certain interval.

In some embodiments, when the elements selected from the group are health-related interventions: a first endpoint of the interval represents no detectable eradication of the adverse health-related condition; and a second endpoint of the interval represents complete eradication of the adverse health-related condition resulting from the health-related interventions.

In some embodiments, when the elements selected from the group are health-related risk factors: a first endpoint of the interval represents no detectable increase of the adverse health-related condition; and numbers which are larger than the first endpoint represent a degree of increase in the adverse health-related condition resulting from the health-related risk factors.

In some embodiments, one or more elements of the historical sets and/or of the new set comprises multiple sub-elements which are interrelated and are therefore deemed to have additive impact.

In some embodiments of the method, it is performed by at least one hardware processor.

In addition to the exemplary aspects and embodiments described above, further aspects and embodiments will become apparent by reference to the figures and by study of the following detailed description.

### BRIEF DESCRIPTION OF THE FIGURES

Exemplary embodiments are illustrated in referenced figures. Dimensions of components and features shown in the figures are generally chosen for convenience and clarity of presentation and are not necessarily shown to scale. The figures are listed below.

FIG. 1 is a block diagram of an exemplary system for predicting a temporal impact of a proposed set of interventions and/or risk factors.

FIG. 2 is a flowchart of a method for predicting a temporal impact of a proposed set of interventions and/or risk factors.

FIG. 3 is a graph illustrating a time series of historical data.

FIG. 4 is a graphical illustration of a sigmoid function descriptive of a typical temporal impact of one constituent out of many in the time series of FIG. 3.

FIG. 5 is a graph illustrating a normalized version of the graph of FIG. 3.

FIG. 6 is a graph of an error function used to model a sigmoid function.

FIG. 7 is a graph of the error function of FIG. 6 following flipping, shifting, and compression.

FIG. 8 is a graph of the error function of FIG. 7 following scaling and shifting.

FIG. 9 is a graph of the error function of FIG. 8 following a time window change.

### DETAILED DESCRIPTION

Disclosed herein is a technique, embodied in a method, a system, and a computer program product, for predicting a temporal impact of a proposed set of interventions and/or risk factors (in fields such as health, industrial processes, agriculture, and more) by learning from the historical temporal impact of other sets of interventions and/or risk factors.

More specifically, the technique is applicable to scenarios in which the proposed set of interventions and/or risk factors has never been implemented and tested before, but only certain subsets thereof have. The technique, accordingly, intelligently deconvolves the temporal impact of each of the historical sets, to determine an individual contribution of each intervention and/or risk factor to the temporal impact of their respective historical sets. Then, once these individual contributions are known, the contributions of the interventions and/or risk factors included in the proposed set may be combined (such as by addition and/or multiplication, as applicable) in order to predict the joint temporal impact of this proposed set.

For example, in the epidemiology field, health authorities are often faced with the need to predict the temporal impact of a new set of proposed preventive measures, such as partial or full lockdowns, mask mandates, vaccinations, social distancing, etc., which have never been implemented simultaneously before in that specific combination. If historical empirical data (collected from the same country and/or other countries) only shows the impact of other combinations of preventive measures but not of the exact proposed combination, it may not be possible to accurately predict how the proposed combination will temporally affect the pandemic’s progress. With the present technique, the temporal impacts of the individual preventive measures may be intelligently deduced from the historical empirical data, such that the joint temporal impact of the proposed set of measures can be predicted by combining individual temporal impacts of the relevant measures. Moreover, the present technique may also allow to factor in various risk factors of the population, such as comorbidities, age, smoking, etc., and to utilize historical data which addressed these risk factors in order to enhance the prediction of the temporal impact of the proposed set of interventions.

As another example, in the industrial processes field, factories and other industrial establishments are sometimes interested in implementing a new set of changes to a certain industrial process, such as to increase or decrease the amount of participating chemicals, to change environmental parameters (such as temperature, humidity, etc.), or to affect other parameters of the process. While the factory may have accumulated historical data on the impact of many process variations tested in the past, there may still be some doubt as to the impact of implementing the new, previously-unseen, set of changes. The present technique may help deconvolve any sets of previously-tested variations, and subsequently predict the impact of implementing the new set of changes.

A further example is from the agriculture field, where crop yields are affected by a multitude of factors, including weather, watering, use of different fertilizers, etc. The present technique may help deconvolve any sets of previously-seen combinations of such factors, and subsequently predict the impact on crop yield by implementing different regimes and/or given changing weather effects.

Those of skill in the art will recognize additional uses of the present technique; the technique may generally be applicable to any scenario in which it is desired to predict the temporal impact of a new set of changes, preventive measures, parameters, etc. (jointly referred to herein as “interventions”), optionally together with certain preexisting risk factors (such as health-related risk factors, weather-related risk factors, etc.), but no historical data of the impact of exactly the same set of interventions and/or risk factors exists. In such scenarios, the deconvolution offered by the present technique, and the subsequent prediction of the impact of the new set, offer a good solution to the problem.

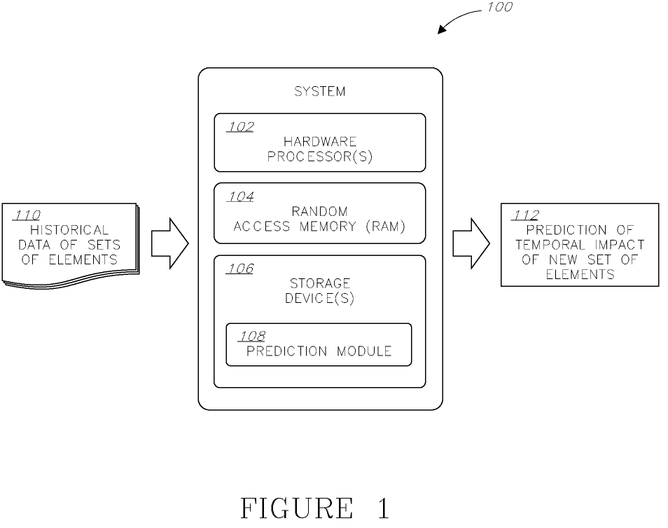

Reference is now made to FIG. 1, which shows a block diagram of an exemplary system 100 for predicting a temporal impact of a proposed set of interventions and/or risk factors, according to an embodiment. System 100 may include one or more hardware processor(s) 102, a random-access memory (RAM) 104, and one or more non-transitory computer-readable storage device(s) 106.

Storage device(s) 106 may have stored thereon program instructions and/or components configured to operate hardware processor(s) 102. The program instructions may include one or more software modules, such as a prediction module 108. The software components may include an operating system having various software components and/or drivers for controlling and managing general system tasks (e.g., memory management, storage device control, power management, etc.), and facilitating communication between various hardware and software components.

System 100 may operate by loading instructions of prediction module 108 into RAM 104 as they are being executed by processor(s) 102. The instructions of prediction module 108 may cause system 100 to receive historical data 110 comprised of sets of interventions and/or risk factors and their temporal impacts, process this data, and output a prediction of the temporal impact of a new set of interventions and/or risk factors 112.

System 100 as described herein is only an exemplary embodiment of the present invention, and in practice may be implemented in hardware only, software only, or a combination of both hardware and software. System 100 may have more or fewer components and modules than shown, may combine two or more of the components, or may have a different configuration or arrangement of the components. System 100 may include any additional component enabling it to function as an operable computer system, such as a motherboard, data busses, power supply, a network interface card, a display, an input device (e.g., keyboard, pointing device, touch-sensitive display), etc. (not shown). Moreover, components of system 100 may be co-located or distributed, or the system may be configured to run as one or more cloud computing “instances,” “containers,” “virtual machines,” or other types of encapsulated software applications, as known in the art.

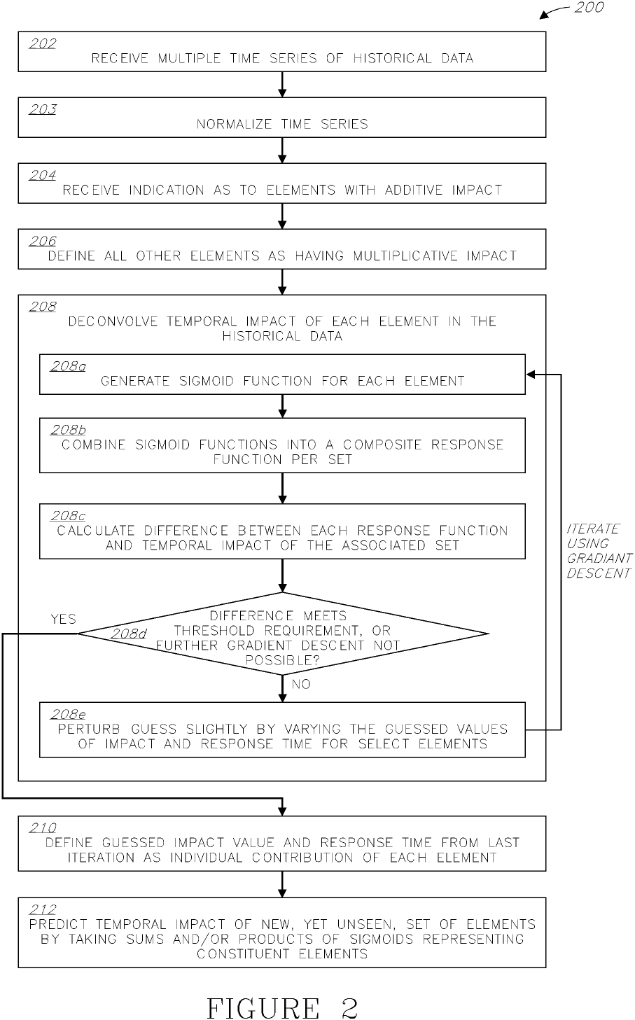

The instructions of prediction module 108 are now discussed with reference to the flowchart of FIG. 2, which illustrates a method 200 for predicting a temporal impact of a proposed set of interventions and/or risk factors, according to an embodiment, in accordance with an embodiment.

Steps of method 200 may either be performed in the order they are presented or in a different order (or even in parallel), as long as the order allows for a necessary input to a certain step to be obtained from an output of an earlier step. In addition, the steps of method 200 are performed automatically (e.g., by system 100 of FIG. 1), unless specifically stated otherwise.

In addition, the discussion of method 200 often makes use of health-related examples in order to simplify its understanding. For instance, much of the discussion exemplifies steps of method 200 by referring to health-related interventions and/or health-related risk factors that are being considered in the context of a global pandemic. Still, these discussions should be interpreted as also encompassing all other types of actions (equivalent to “interventions”) and/or related risk factors to which the present technique is applicable.

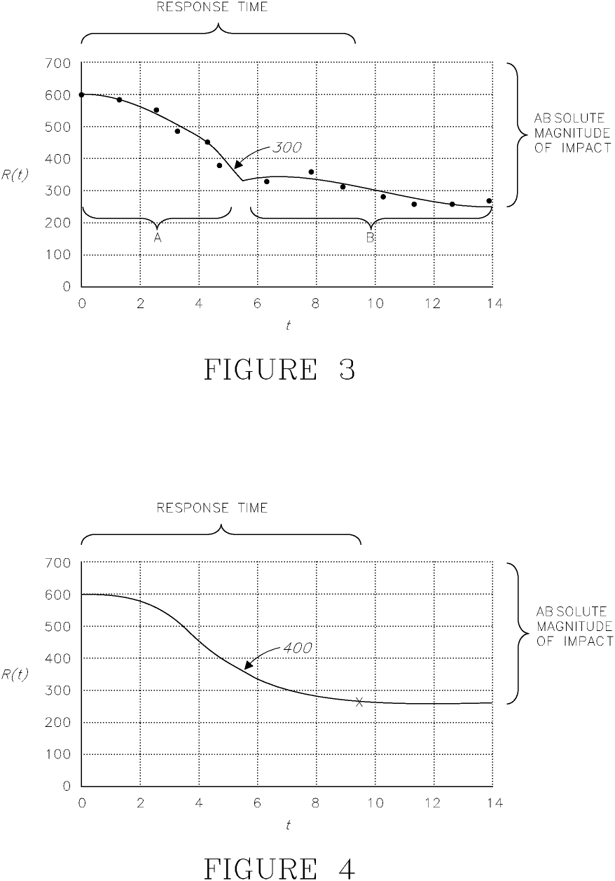

In a step 202, multiple time series of historical data may be received. Each of these time series, referred to herein as a “set”, may include values recorded at distinct points in time. For example, with interim reference to FIG. 3 which illustrates such time series graphically, each such time series (“set”) may include values (shown as round dots) of numbers of pandemic-resultant hospitalizations per million people at different dates during a fourteen-week period which follows implementation of one or more measures. These measures are referred to herein as “elements” of the set, and may include, e.g., health-related interventions and/or health-related risk factors. Time is denoted t, and the number of hospitalizations is denoted R(t), and may be thought of as a response rate as a function of time. Here R(0) is the number of hospitalizations at time t=0. As the interventions (“elements”) start taking effect, R(t) declines. The set of measures may include, for example, closure of all entertainment establishments (e.g., theaters, event halls, etc.), closure of all dining establishments (e.g., restaurants and bars), a mask mandate outside one’s home, and public guidance for a 6-foot social distancing.

Clearly, it is impossible to deduce, based on this data only, the individual impact (also referred to herein as “effect” or “result”) of each of these elements in the set on the number of hospitalizations, as they were all implemented concurrently.

It can also be observed, in this example, that the time series approximates a somewhat amorphic curve 300, which cannot be readily categorized as sigmoid, sine, polynomial, linear, or the like. Still, it is possible to see that two somewhat distinct time windows, denoted A and B in FIG. 3, do exist. Each of these time windows exhibit a different response: Time window A shows a relatively fast response to the implemented measures, while time window B shows a much more moderate response, which could even be seen as a sort of plateau.

Interim reference is now made to FIG. 4. In many fields, it is known that each individual element in such set of measures tends to approximate a sigmoid function (more specifically, an inverse sigmoid function) 400, as this Figure illustrates. For example, a single health-related measure that is being taken to combat a spreading pandemic (such as a mask mandate) seems to have little to no effect early on (for example, for the first couple of weeks, as shown in this Figure), following which its effect exponentially increases (for example, between weeks 2 and 5), and then begins to slow down and eventually plateaus (for example, following week 9) as the masks have reached their full preventative potential.

The reason for the shape difference between the approximate functions in FIGS. 3 and 4 is that, even if the underlying elements in the set of FIG. 3 each approximate a sigmoid, the convolution (combination) of different sigmoids (having vastly different value distributions across the X and Y axes) is unlikely to result in a sigmoid. This is much like the reason why the convolution of sine waves having different frequencies and amplitudes will almost never look like a sine wave. A further reason for the disparity between these approximate functions is that some elements may have an additive effect when implemented together, while others may have a multiplicative effect when implemented together. This further deforms the structure of the convolved function.

To summarize step 202 of FIG. 2, each of the received time series of data (each “set”) may represent a health-related temporal impact of a historical set of elements (e.g., health-related interventions, and/or health-related risk factors) on an adverse health-related condition, such as a pandemic. All these sets, together, may be simply referred to as “historical data.” It should be noted that each element may in fact include multiple sub-elements which are interrelated and have always existed together in whichever historical sets they were included. For example, if closure of elementary schools always appeared together with closure of high schools in the historical data, then these measures may be regarded as a single element for purposes of method 200. They may also be deemed to have additive impact.

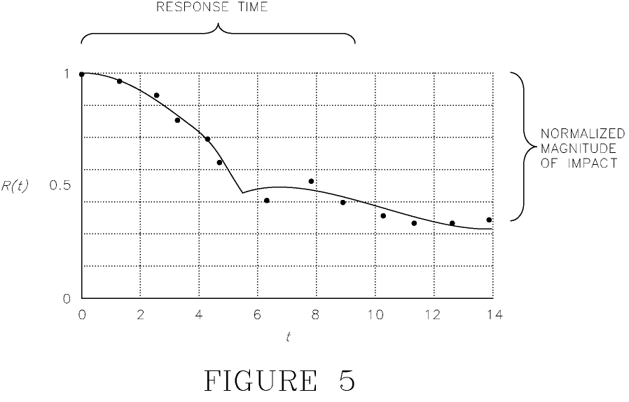

In a step 203, each of the received time series of data are normalized to fall in [0,1], for example. More specifically, the impact values are the ones being normalized with the value 1 corresponding to the value of the variable being measured at the initiation of the intervention (e.g., the number of hospitalizations), while the time values may remain the same (e.g., on a scale of days, weeks, or months). Interim reference is now made to FIG. 5, which illustrates the same exemplary time series of FIG. 3 but, schematically, in a normalized form. As shown on the Y axis, a first endpoint of the interval (‘R(0) = 1’ in this example) is where the response (R(t)) to the health-related measures begins, namely, at t = 0. At this endpoint, no detectable eradication of the adverse health-related condition can yet be seen. The maximum visible magnitude of impact is approximately 0.3, meaning an approximately 70% decrease in the associated adverse effect, as shown, and the maximal theoretical magnitude (not reached in this example) is the value ‘0,’ representing complete eradication of the adverse health-related condition as a result of the health-related intervention.

When risk factors are considered (as opposed to measures, or ‘interventions’), the normalization may be performed such that a first endpoint of the interval (again, ‘R(0) = 1’) represents no detectable increase of the adverse health-related condition as a result of the particular risk factor, while a second endpoint of the interval (which could be any number, r >1) represents a degree (e.g., a percentage) of increase in the adverse health-related condition resulting from the health-related risk factor. For example, smoking is a well-known risk factor in viral respiratory diseases. If, according to the historical data, smoking doubles the risk of hospitalization in a given time period, then the degree of increase would be set to 2. The same rationale applies to other risk factors, both in health-related scenarios as well as in industrial, agricultural, and other scenarios.

The subsequent steps of method 200, following the normalization step 203, are performed on the normalized values of the received time series instead of on the original values.

Back to FIG. 2, in a step 204, an indication may be received (e.g., from a user) as to whether there are two or more elements in any of the historical data that are estimated to jointly have additive impact when the elements appear together in any of the sets of the historical data. For example, if the user estimates that that closure of entertainment establishments (e.g., theaters, event halls, etc.) and dining establishments (e.g., restaurants and bars) are two closely-related measures that together simply decrease the overall number of people gathering, then the user may define these measures as having an additive impact. This differentiates these measures from those which have multiplicative impact, as will be discussed in the next step. It should be noted that the indication does not have to separately address each set in the historical data; rather, it may simply link elements that have an additive effect, no matter where they exist in the historical data. To this end, the user may be presented with a list of all elements existing in all historical data, and be asked to define or group together elements with an additive impact between them. This may lead to the creation of multiple such groups.

In a step 206, all elements which were not selected in step 204 as having an additive impact may be defined as elements that have multiplicative impact. For example, if the user did not select dining establishment closure and mask mandate as having a joint additive impact, then step 206 may automatically define these two elements as having a multiplicative impact.

The rationale behind steps 204 and 206 is that the additive or multiplicative relationships between elements changes the way they are deconvolved. This is discussed below in further detail.

As an alternative to steps 204 and 206, if it is known in advance that all elements existing in the historical data have the same type of impact (additive or multiplicative), then method 200 may simply be pre-programmed to treat all elements as having that type of impact without having to seek a specific indication from the user.

In a step 208, the health-related temporal impact of each element appearing in any of the historical data is deconvolved, in order to determine an individual contribution of each element. The deconvolution may include applying gradient descent optimization to the health-related temporal impact, by performing the following sub-steps:

In a sub-step 208a, for each element (e.g., a health-related intervention) in the historical data, a sigmoid function may be generated, which is based on a guessed impact value and a guessed response time value of the respective element. Reference is now made back to FIG. 4, to illustrate what these guessed impact value and response time value mean in the context of a sigmoid function representing such temporal impact. In the context of sigmoid function 400, the guessed impact value (denoted, in the Figure, as the “absolute magnitude of impact”) and response time value (denoted, in the Figure, as the “response time”) are essentially a point on that function’s graph, marked with an X in this Figure, in which the near-maximal impact of the pertinent health-related measure has been reached. It is possible to define this point, for example, as the first point on the graph for which the first derivative has a certain predefined value, close to zero, indicating that the slope of the function is nearly flat (e.g., the value of t such that |R′(t)| < 0.05) and the second derivative R′(t)>0, discounting the early points on the graph, for which the second derivative is negative.. By generating a guessed value of this point (such as using a random or pseudo-random algorithm), a guessed (randomly or pseudo-randomly) sigmoid function can be produced.

Mathematically, to generate the sigmoid function for each of the elements, the following technique may be used (in which time is denoted t, the value of the maximum [or near-maximum] impact is denoted r, and the time window [also ‘response time’] to reach r is denoted w):

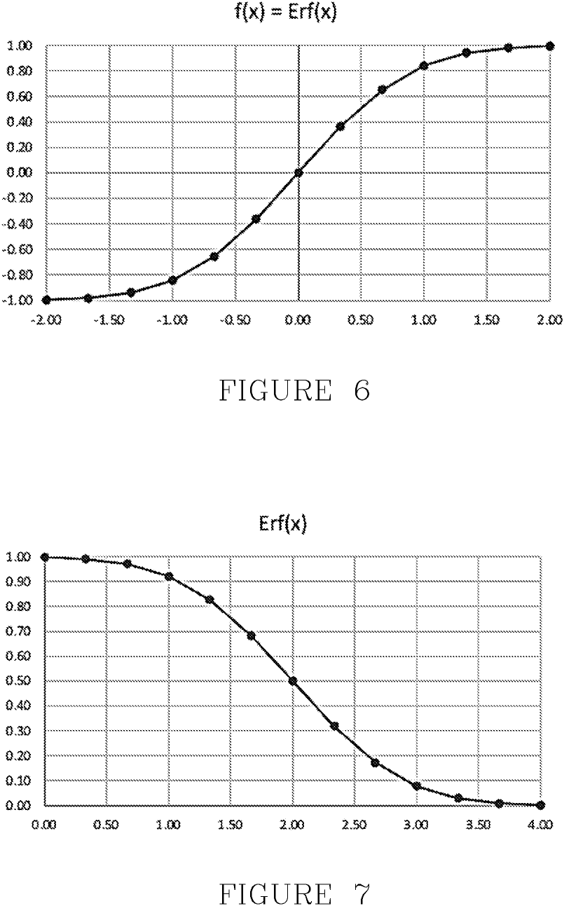

The sigmoid function may be first modeled as the following error function, illustrated graphically in FIG. 6:

$$f \left(x\right) = \text{erf} \left(x\right) = \frac{2}{\sqrt{π}} {\int }_{0}^{x} {e}^{- {t}^{2}} d t , \text{for} x ∈ \left[- 2 , 2\right]$$

It is also, possible, however, to start with a logistic function or any other sigmoid function.

Then, the error function may be flipped about the X-axis by ƒ(x) = -erf(x), and shifted by two units so that the vast majority of the mass of the flipped function starts at x = 0. These modifications yield:

$$f \left(x\right) = - \text{erf} \left(x - 2\right) = \frac{2}{\sqrt{π}} {\int }_{0}^{x - 2} {e}^{- {t}^{2}} d t , \text{for} x ∈ \left[0 , 4\right]$$

Next, the function may be shifted up by one unit in the Y direction, so that it ranges from [0,2] rather than [-1,1], and then compressed by half, yielding, as graphically illustrated in FIG. 7:

$$f \left(x\right) = - \frac{1}{2} \text{erf} \left(x - 2\right) + \frac{1}{2} = \frac{1}{\sqrt{π}} {\int }_{0}^{x - 2} {e}^{- {t}^{2}} d t + \frac{1}{2} , \text{for} x ∈ \left[0 , 4\right]$$

The function may then be scaled so that x ∈ [0,4] and y ∈ [0,1]. To get the function’s graph to plateau at a given y = r rather than y = 0, it may be first scaled to [0,1-r] and then shifted up by r, yielding, as graphically illustrated in FIG. 8:

$$f \left(x\right) = \left(\frac{1 - r}{\sqrt{π}} {\int }_{0}^{x - 2} {e}^{- {t}^{2}} d t + \frac{1 - r}{2}\right) + \text{r} , \text{for} x ∈ \left[0 , 4\right]$$

$$f \left(x\right) = \frac{1 - r}{\sqrt{π}} {\int }_{0}^{x - 2} {e}^{- {t}^{2}} d t + \frac{1 + r}{2} , \text{for} x ∈ \left[0 , 4\right]$$

Next, to change the time window from [0,4] to [0,w], the variable x may be simply changed to

$$\frac{4 x}{w} ,$$

and then for

$$x ∈ \left[0 , w\right] , \frac{4 x}{w} ∈ [ 0 , 4 ] ,$$

so:

$$f \left(x\right) = \frac{1 - r}{\sqrt{π}} {\int }_{0}^{\frac{4 x}{w} - 2} {e}^{- {t}^{2}} d t + \frac{1 + r}{2} , \text{for} x ∈ \left[0 , w\right]$$

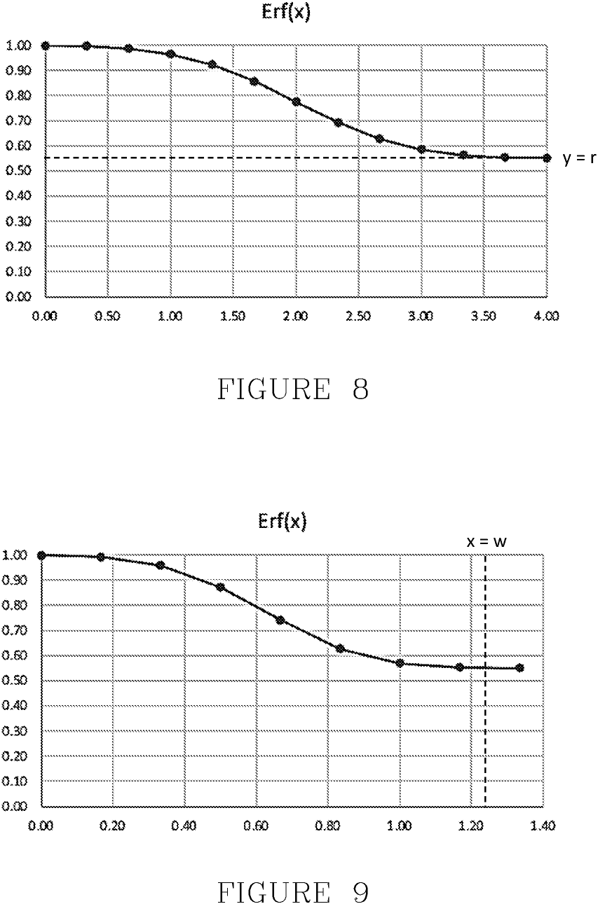

as graphically illustrated in FIG. 9.

Next, if all elements (interventions) contributed multiplicatively, a “family” of sigmoids would be produced, each with two parameters, ƒᵢ(rᵢ,wᵢ). Now, suppose there is a group of elements (interventions) that together act additively. Such group may be regarded as a “sub-family,” and its sigmoids are additive. However, once the additive elements are summed, the resultant aggregate contributes multiplicatively with respect to the other interventions.

Referring back to FIG. 2, once sub-step 208a has been performed for each element in the historical data, the result is a plurality of sigmoid functions, one per element.

In a sub-step 208b, the sigmoid functions of all elements in each set may be combined into a composite response function which represents that set. This is performed for all the sets in the historical data. The combining may include multiplication and/or addition of the sigmoid functions generated (for the elements of the respective historical set) in sub-step 208a. The multiplication and/or addition correspond to the multiplicative impact and the additive impact, respectively. This ensures that the deconvolution in the entirety of step 208 is performed respectively of the aforementioned additive impact and multiplicative impact possibilities.

The multiplication and/or addition of sub-step 208b may be performed pointwise at every point in time for which there is historical data associated with the given set.

Next, in a sub-step 208c, a difference between each composite response function produced in sub-step 208b and the health-related temporal impact of the respective historical set received in step 202 may be calculated, in order to quantify the similarity between that composite response function, which is the result of a guess, and the real historical data. When this difference is small, it means that the composite response function generated in sub-step 208a is close to the true temporal impact of the combined effect of the elements in the respective set. This process is conducted for each set, and the difference is computed for all historical sets and then summed to get a total “error”. This cumulative error may be computed as a cumulative absolute error, cumulative sum of squares error, or by any other means.

Then, in a sub-step 208d, a determination is made as to whether the difference calculated in sub-step 208c meets a predefined threshold requirement, and/or whether further gradient descent is not possible. As to the predefined threshold requirement, this may be a numerical value of a small enough error between each composite response function and its associated temporal impact in the historical data, and/or a numerical value of a small enough total error, as described above.

In case it is determined in sub-step 208d that the predefined threshold requirement is not yet met, the method may proceed to a step 208e, in which the guessed impact value and the guessed response time value of step 202 are slightly perturbed using gradient descent. Then, to reach a sufficiently minimal difference (or to reach a state in which gradient descent is no longer possible), sub-steps 208a-208e may be iterated, with a different guessed impact value and a different guessed response time value (as perturbed in sub-step 208e) in each iteration. Such iteration yields different sigmoid functions in sub-step 208a, a different composite response function in sub-step 208b, and a different calculated difference in sub-step 208c.

In a step 210, once one or both of these alternative threshold requirements is met, or further gradient descent is no longer possible, the guessed impact value and the guessed response time of the last iteration may be defined as the determined individual contribution of each element of the historical data to the pertinent health-related temporal impact. Put differently, this happens when each of the composite sigmoid functions of sub-step 208b is found to be as true as possible to its respective historical sets, and thus justifies treating the guessed impact value and response time from the last iteration as the ones which best explain the individual contributions of the elements of the respective historical sets.

Next, in a step 212, it is possible to predict a temporal impact of the new set of elements that have never been implemented before in the same exact combination. Namely - the new set is different from any of the historical sets; it consists of elements which do not jointly constitute any one of the historical sets.

The prediction of step 212 may be performed based on the individual contributions defined in step 210, by adding and/or multiplying these individual contributions respective of their definition as additive or multiplicative in steps 204 and 206.

In conclusion, method 200 provides an advantageous way to deconvolve the temporal impact of historical data that is provided as multiple sets of time series, to determine an individual contribution of each element in the historical data to the temporal impact of historical sets in which it appeared. Then, once these individual contributions are known, the contributions of elements in a newly-proposed set may be combined (such as by addition and/or multiplication, as applicable) in order to predict the joint temporal impact of this proposal.

The present invention may be a system, a method, and/or a computer program product. The computer program product may include a computer readable storage medium (or media) having computer readable program instructions thereon for causing a hardware processor (e.g., a CPU, a GPU, etc.) to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire. Rather, the computer readable storage medium is a non-transient (i.e., not-volatile) medium.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object-oriented programming language such as Java, Smalltalk, C++ or the like, and conventional procedural programming languages, such as the “C” programming language or similar programming languages. The computer readable program instructions may execute entirely on the user’s computer, partly on the user’s computer, as a stand-alone software package, partly on the user’s computer and partly on a remote computer or entirely on the remote computer or server. In the latter scenario, the remote computer may be connected to the user’s computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, a field-programmable gate array (FPGA), or a programmable logic array (PLA) may execute the computer readable program instructions by utilizing state information of the computer readable program instructions to personalize the electronic circuitry, in order to perform aspects of the present invention. In some embodiments, electronic circuitry including, for example, an application-specific integrated circuit (ASIC), may be incorporate the computer readable program instructions already at time of fabrication, such that the ASIC is configured to execute these instructions without programming.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

In the description and claims, each of the terms “substantially,” “essentially,” and forms thereof, when describing a numerical value, means up to a 20% deviation (namely, ±20%) from that value. Similarly, when such a term describes a numerical range, it means up to a 20% broader range - 10% over that explicit range and 10% below it).

In the description, any given numerical range should be considered to have specifically disclosed all the possible subranges as well as individual numerical values within that range, such that each such subrange and individual numerical value constitutes an embodiment of the invention. This applies regardless of the breadth of the range. For example, description of a range of integers from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 4, and 6. Similarly, description of a range of fractions, for example from 0.6 to 1.1, should be considered to have specifically disclosed subranges such as from 0.6 to 0.9, from 0.7 to 1.1, from 0.9 to 1, from 0.8 to 0.9, from 0.6 to 1.1, from 1 to 1.1 etc., as well as individual numbers within that range, for example 0.7, 1, and 1.1.

The descriptions of the various embodiments of the present invention have been presented for purposes of illustration, but are not intended to be exhaustive or limited to the explicit descriptions. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

In the description and claims of the application, each of the words “comprise,” “include,” and “have,” as well as forms thereof, are not necessarily limited to members in a list with which the words may be associated.

Where there are inconsistencies between the description and any document incorporated by reference or otherwise relied upon, it is intended that the present description controls.

What is claimed is:

1. A computer-implemented method comprising:
receiving multiple time series of data, each time series of data representing a health-related temporal impact of a historical set of elements on an adverse health-related condition, wherein the elements are selected from the group consisting of: health-related interventions, and health-related risk factors;
receiving an indication of whether there are two or more elements in any of the historical sets that are estimated to jointly have additive impact within the respective set;
automatically defining all non-selected elements as elements that have multiplicative impact within their respective sets;
automatically deconvolving the health-related temporal impact of each of the historical sets, to determine an individual contribution of each element of each of the historical sets to the health-related temporal impact, wherein said deconvolving is performed respectively of the additive impact and multiplicative impact;
receiving a selection of a new set of elements which is different from any one of the historical sets; and
based on the determined individual contributions, automatically predicting a temporal impact of the new set of elements, wherein the new set consists of elements selected from said group but which do not jointly constitute any one of the historical sets.

2. The computer-implemented method of claim 1, wherein:
said deconvolving comprises applying gradient descent optimization to the health-related temporal impact of each of the historical sets, by:
(a) for each element in the respective historical set, generating a sigmoid function which is based on a guessed impact value and a guessed response time value of the respective element,
(b) combining the sigmoid functions of all elements in the respective set into a composite response function which represents the respective set, wherein said combining comprises multiplication and addition of the sigmoid functions, in correspondence to the multiplicative impact and the additive impact, respectively,
(c) calculating a difference between the composite response function and the received health-related temporal impact of the respective historical set,
(d) iterating steps (a) through (c) with a different guessed impact value and a different guessed response time value in each iteration, until the calculated difference is reduced to or below a predetermined threshold, and/or until further gradient descent is no longer possible,
(e) defining the different guessed impact value and the different guessed response time of the last iteration as the determined individual contribution of each element of the respective historical set to the health-related temporal impact of the respective historical set.

3. The computer-implemented method of claim 2, wherein:
said predicting of the temporal impact of the new set of elements comprises multiplying and/or adding the determined individual contributions of those elements included in the new set of elements, wherein the multiplying and/or adding correspond to the multiplicative impact and/or the additive impact, respectively.

4. The computer-implemented method of claim 2, further comprising:
prior to said deconvolving, automatically normalizing the multiple time series of data to a certain interval.

5. The computer-implemented method of claim 4, wherein, when the elements selected from the group are health-related interventions:
a first endpoint of the interval represents no detectable eradication of the adverse health-related condition; and
a second endpoint of the interval represents complete eradication of the adverse health-related condition resulting from the health-related interventions.

6. The computer-implemented method of claim 4, wherein, when the elements selected from the group are health-related risk factors:
a first endpoint of the interval represents no detectable increase of the adverse health-related condition; and
numbers which are larger than the first endpoint represent a degree of increase in the adverse health-related condition resulting from the health-related risk factors.

7. The computer-implemented method of claim 1, wherein:
one or more elements of the historical sets and/or of the new set comprises multiple sub-elements which are interrelated and are therefore deemed to have additive impact.

8. The computer-implemented method of claim 1, performed by at least one hardware processor.

9. A system comprising:
(i) at least one hardware processor; and
(ii) a non-transitory computer-readable storage medium having program code embodied therewith, the program code executable by said at least one hardware processor to:
receive multiple time series of data, each time series of data representing a health-related temporal impact of a historical set of elements on an adverse health-related condition, wherein the elements are selected from the group consisting of: health-related interventions, and health-related risk factors;
receive an indication of whether there are two or more elements in any of the historical sets that are estimated to jointly have additive impact within the respective set;
automatically define all non-selected elements as elements that have multiplicative impact within their respective sets;
automatically deconvolve the health-related temporal impact of each of the historical sets, to determine an individual contribution of each element of each of the historical sets to the health-related temporal impact, wherein said deconvolving is performed respectively of the additive impact and multiplicative impact;
receive a selection of a new set of elements which is different from any one of the historical sets; and
based on the determined individual contributions, automatically predict a temporal impact of the new set of elements, wherein the new set consists of elements selected from said group but which do not jointly constitute any one of the historical sets.

10. The system of claim 9, wherein:
said deconvolving comprises applying gradient descent optimization to the health-related temporal impact of each of the historical sets, by:
(a) for each element in the respective historical set, generating a sigmoid function which is based on a guessed impact value and a guessed response time value of the respective element,
(b) combining the sigmoid functions of all elements in the respective set into a composite response function which represents the respective set, wherein said combining comprises multiplication and addition of the sigmoid functions, in correspondence to the multiplicative impact and the additive impact, respectively,
(c) calculating a difference between the composite response function and the received health-related temporal impact of the respective historical set,
(d) iterating steps (a) through (c) with a different guessed impact value and a different guessed response time value in each iteration, until the calculated difference is reduced to or below a predetermined threshold, and/or until further gradient descent is no longer possible,
(e) defining the different guessed impact value and the different guessed response time of the last iteration as the determined individual contribution of each element of the respective historical set to the health-related temporal impact of the respective historical set.

11. The system of claim 10, wherein:
said predicting of the temporal impact of the new set of elements comprises multiplying and/or adding the determined individual contributions of those elements included in the new set of elements, wherein the multiplying and/or adding correspond to the multiplicative impact and/or the additive impact, respectively.

12. The system of claim 10, wherein said program code is further executable to:
prior to said deconvolving, automatically normalize the multiple time series of data to a certain interval.

13. The system of claim 12, wherein, when the elements selected from the group are health-related interventions:
a first endpoint of the interval represents no detectable eradication of the adverse health-related condition; and
a second endpoint of the interval represents complete eradication of the adverse health-related condition resulting from the health-related interventions.

14. The system of claim 12, wherein, when the elements selected from the group are health-related risk factors:
a first endpoint of the interval represents no detectable increase of the adverse health-related condition; and
numbers which are larger than the first endpoint represent a degree of increase in the adverse health-related condition resulting from the health-related risk factors.

15. The system of claim 9, wherein:
one or more elements of the historical sets and/or of the new set comprises multiple sub-elements which are interrelated and are therefore deemed to have additive impact.

16. A computer program product comprising a non-transitory computer-readable storage medium having program code embodied therewith, the program code executable by at least one hardware processor to:
receive multiple time series of data, each time series of data representing a health-related temporal impact of a historical set of elements on an adverse health-related condition, wherein the elements are selected from the group consisting of: health-related interventions, and health-related risk factors;
receive an indication of whether there are two or more elements in any of the historical sets that are estimated to jointly have additive impact within the respective set;
automatically define all non-selected elements as elements that have multiplicative impact within their respective sets;
automatically deconvolve the health-related temporal impact of each of the historical sets, to determine an individual contribution of each element of each of the historical sets to the health-related temporal impact, wherein said deconvolving is performed respectively of the additive impact and multiplicative impact;
receive a selection of a new set of elements which is different from any one of the historical sets; and
based on the determined individual contributions, automatically predict a temporal impact of the new set of elements, wherein the new set consists of elements selected from said group but which do not jointly constitute any one of the historical sets.

17. The computer program product of claim 16, wherein:
said deconvolving comprises applying gradient descent optimization to the health-related temporal impact of each of the historical sets, by:
(a) for each element in the respective historical set, generating a sigmoid function which is based on a guessed impact value and a guessed response time value of the respective element,
(b) combining the sigmoid functions of all elements in the respective set into a composite response function which represents the respective set, wherein said combining comprises multiplication and addition of the sigmoid functions, in correspondence to the multiplicative impact and the additive impact, respectively,
(c) calculating a difference between the composite response function and the received health-related temporal impact of the respective historical set,
(d) iterating steps (a) through (c) with a different guessed impact value and a different guessed response time value in each iteration, until the calculated difference is reduced to or below a predetermined threshold, and/or until further gradient descent is no longer possible,
(e) defining the different guessed impact value and the different guessed response time of the last iteration as the determined individual contribution of each element of the respective historical set to the health-related temporal impact of the respective historical set.

18. The system of claim 17, wherein:
said predicting of the temporal impact of the new set of elements comprises multiplying and/or adding the determined individual contributions of those elements included in the new set of elements, wherein the multiplying and/or adding correspond to the multiplicative impact and/or the additive impact, respectively.

19. The system of claim 17, wherein said program code is further executable to:
prior to said deconvolving, automatically normalize the multiple time series of data to a certain interval.

20. The system of claim 19, wherein:
when the elements selected from the group are health-related interventions:
a first endpoint of the interval represents no detectable eradication of the adverse health-related condition, and
a second endpoint of the interval represents complete eradication of the adverse health-related condition resulting from the health-related interventions; and
when the elements selected from the group are health-related risk factors:
a first endpoint of the interval represents no detectable increase of the adverse health-related condition, and
numbers which are larger than the first endpoint represent a degree of increase in the adverse health-related condition resulting from the health-related risk factors.
